(19) United States
(12) Patent Application Publication
(10) Pub. No.: US 2016/0008057 A1
(43) Pub. Date: Jan. 14, 2016

(54) DIAGNOSTIC NEEDLE PROBE

(71) Applicant: EMPIRE TECHNOLOGY DEVELOPMENT LLC, Wilmington, DE (US)

(72) Inventor: George Charles PEPPOU, Hornsby Heights (AU)

(73) Assignee: EMPIRE TECHNOLOGY DEVELOPMENT LLC, Wilmington, DE (US)

(21) Appl. No.: 14/770,580

(22) PCT Filed: Feb. 27, 2013
(86) PCT No.: PCT/US13/27952

§ 371 Date: Aug. 26, 2015

(51) Int. Cl. A61B 18/14; A61B 5/00; A61B 5/1455; A61B 18/02

(52) U.S. Cl. CPC A61B 18/1477; A61B 18/02; A61B 5/0084; A61B 5/14552; A61B 2018/0293; A61B 2018/00577

(57) ABSTRACT

Properties of biological tissue may be determined percutaneously and intraoperatively with a needle probe which includes a number of sets of emitting and collecting optical fibers terminating at different locations along the length of the needle. Such an optical fiber arrangement enables tissue information to be gathered across the entire needle length, allowing for the rapid provision of information about the tissue surrounding the needle probe at several positions along the probe.

### BACKGROUND

Cells are the building blocks of living things and form the tissues and organs of the body. Normal cells multiply when the body needs them, follow a regular growth cycle and die when the body doesn't need them. Cancer grows out of normal cells in the body. Gene damage, as well as other causes, can alter the cells, resulting in cancerous cells growing among the non-cancerous cells. Cancer appears to occur when the growth of cells in the body is out of control and cells divide too quickly. It can also occur when cells forget how to die. Cancer can take the form of solid tumors, lymphomas and non-solid cancers such as leukemia

Cancer cells generally have different characteristics and properties as compared to normal, or healthy cells which surround the cancer cells, and because of the differences, visual resection of tumors is often possible. Resection of tumors remains a successful method of treatment for a large number of patients with solid tumor masses, however incomplete resection, caused by inadequate margins of healthy tissue being removed may require a second surgery to be performed to remove additional tissue, and may also be responsible for a recurrence of the disease.

Verification of a healthy margin is generally achieved through pathology results, a process which may take hours or days, so that the results may not be available until the patient is out of surgery. In the event an incomplete resection is observed, the patient may therefore require a second surgery, resulting in a significant increase in the overall cost of care. There remains a need for an improved detection system which may provide essentially instantaneous results during a surgical procedure.

Devices for monitoring, measuring, or diagnosing a physiological condition or a biological phenomenon may be used to quickly evaluate a condition or detect a phenomenon by using spectrophotometry. A number of procedures for monitoring or diagnosing medical conditions benefit from the ability to use spectrometric means to accomplish the procedure. For example, pulse oximeters use spectrophotometry to determine oxygen saturation of blood. In general, when radiant energy passes through a liquid and/or tissue, certain wavelengths may be selectively absorbed by cellular contents present therein, and the absorption may be used for determining a feature or quality of the tissue which may be useful during surgical procedures. However, spectrophotometric analysis is generally done ex vivo and often at a location or lab distant from an operating room where a surgical procedure is being performed, thereby resulting in a delay, often up to a day or more, before results are available.

### SUMMARY

Properties of biological tissue may be determined percutaneously and intraoperatively with a needle probe which includes a one or more sets of emitting and collecting optical fibers terminating at different locations along the length of the needle. Such an optical fiber arrangement, coupled with appropriate monitoring equipment, enables tissue information to be gathered across the entire needle length, allowing for the rapid, on site, provision of information about not only the needle tip location within the tissue, but surrounding tissue as well. This information is particularly relevant for diffuse tumors, whose margins may be imprecise, and challenging to determine by alternative imaging modalities, and may enable improved results in determining tumor margins for such procedures as tumor excision or ablation.

In an embodiment, a method for determining at least one property of biological tissue at different positions within the tissue includes the step of inserting an optical probe into the tissue, wherein the probe includes a shaft having a proximal end, a distal end for being inserted into the tissue, and a plurality of sets of optical fibers disposed circumferentially about an exterior surface of the shaft with terminal ends of each set being disposed at spaced apart intervals relative to the distal end of the probe, and different from a location of the terminal end of any other set. Each set includes at least one optical fiber for emitting light into the tissue and at least one optical fiber for collecting and returning light from within the tissue, with an end of the at last one emitting fiber being disposed circumferentially adjacent an end of the at least one collecting fiber. The method also includes simultaneously transmitting light through each at least one light emitting fiber of each set and out the terminal ends thereof to simultaneously probe the biological tissue adjacent the terminal end of each fiber optic set at the spaced apart intervals, and collecting light adjacent the terminal ends of the light emitting fibers with the adjacent light collecting fibers. The method also includes separately and simultaneously detecting light returned through the at least one light collecting fiber of each set, wherein the light returned has at least one property correlating to the at least one property of the biological tissue, and continuously displaying and updating the at least one property of the detected light from each fiber optic set during the inserting to determine the at least one property of the biological tissue adjacent the corresponding end of each fiber optic set.

In an embodiment, a method for intra-operatively determining the margins of cancerous tissue during resection of the cancerous tissue includes the step of inserting an optical probe to a predetermined depth into biological tissue to pass through a cancerous tissue therein, wherein the probe includes a shaft having an outer cylindrical surface, a proximal end, a distal end for being inserted into the biological tissue, and a plurality of optical fiber sets, wherein each set has a terminal end disposed at a different position relative to the distal end than the terminal end of any other set, and each set includes at least one light emitting fiber and at least one light collecting fiber. The method also includes transmitting light through the light emitting fibers and out the terminal ends thereof into the biological tissue at depths into the biological tissue corresponding to the positions of the ends of the optical fiber sets, and separately detecting light returned through the at least one light collecting fiber of each optical fiber set, wherein the light returned through the at least one light collecting fiber of each optical fiber set includes at least one property indicative of a health of the biological tissue at the corresponding depths into the biological tissue. In addition, the method also includes displaying the at least one property of the detected light from each optical fiber set to depict whether the biological tissue adjacent the end of each optical fiber set is cancerous tissue, precancerous tissue or healthy tissue and provide a display for determining margins of the cancerous tissue. After determining the margins, the method also includes resecting the cancerous tissue and repeating the steps of transmitting, detecting and displaying to determine if any cancerous tissue remains requiring further resection.

In an embodiment, a method for surgical ablation of cancerous tissue from within non-cancerous tissue includes a step of inserting an optical probe into biological tissue having a cancerous tissue portion and a non-cancerous tissue portion, wherein the probe includes a needle having an outer cylindrical surface, an axial bore, a proximal end, and a distal end for being inserted into the biological tissue. The needle includes a plurality of optical fiber sets with each set having a terminal end disposed at the outer cylindrical surface and disposed at a different position relative to the distal end than the terminal end of any other set, and each set comprises at least one light emitting fiber and at least one light collecting fiber. The method also includes transmitting light through the light emitting fibers and out the terminal ends thereof, and separately detecting light returned by the at least one light collecting fiber of each optical fiber set, wherein the returned light has at least one property usable for distinguishing the cancerous tissue from the non-cancerous tissue. In addition, the method also includes determining from the detected light from each optical fiber set, a tissue type adjacent the ends of each optical fiber set to locate cancerous tissue, locating the distal end of the needle within the cancerous tissue, and ablating the cancerous tissue via the axial bore of the needle.

In an embodiment, a method for verifying a tissue margin of an excised tumor, includes inserting an optical probe into the tissue to depth corresponding to at least a thickness of the margin. The probe includes a shaft having an outer cylindrical surface, a proximal end, and a distal end for being inserted into the biological tissue, and a plurality of optical fiber sets with each set having a terminal end disposed at a different position relative to the distal end than the terminal end of any other set, and each set comprises at least one light emitting fiber and at least one light collecting fiber. The method further includes transmitting light through the light emitting fibers and out the terminal ends thereof into the tissue at depths into the tissue corresponding to the positions of the ends of the optical fiber sets, and separately detecting light returned through the at least one light collecting fiber of each optical fiber set. The light returned through the at least one light collecting fiber of each optical fiber set comprises at least one property indicative of a health of the tissue at the corresponding depths into the biological tissue. The method also includes displaying the at least one property of the detected light from each optical fiber set to depict whether the tissue adjacent the end of each optical fiber set is cancerous tissue, precancerous tissue or healthy tissue.

In an embodiment, an optical system for determining at least one property of a material at a plurality of locations within the material, includes a needle probe having a shaft with an outer cylindrical surface, a proximal end, and a distal end for being inserted into the biological tissue. In addition, the needle probe also includes a lumen extending from the proximal end to the distal end and configured for conduction of at least one surgical procedure through the lumen; and a plurality of sets of optical fibers disposed about the shaft. Each set of optical fibers has a terminal end at the outer cylindrical surface and disposed at a different position relative to the distal end than the terminal end of any other set, and each set includes at least one optical fiber for emitting electromagnetic radiation into the material and at least one optical fiber for collecting and returning electromagnetic radiation from within the tissue. The system also includes at least one light source for providing at least a first bandwidth of light to the light transmitting fibers, and at least one detector for receiving returning light from the at least one light collecting fibers and outputting at least one signal corresponding to at least one property of the returning light, wherein the at least one property of the returning light from each fiber optic set correlates with at least one property of the material adjacent the end of each fiber optic set.

In an embodiment, a diagnostic needle for use in determining at least one property of biological tissue at a plurality of locations along the needle includes a shaft having an outer cylindrical surface, a proximal end, and a distal end for being inserted into the biological tissue. The needle also includes a lumen extending from the proximal end to the distal end and being configured for conduction of at least one surgical procedure through the lumen, and a plurality of sets of optical fibers disposed about the shaft. Each set of optical fibers has a terminal end at the outer cylindrical surface and disposed at a different position relative to the distal end than the terminal end of any other set, and each set includes at least one optical fiber for emitting electromagnetic radiation and at least one optical fiber for collecting electromagnetic radiation.

### BRIEF DESCRIPTION OF THE FIGURES

FIG. 1 depicts an optical needle probe according to an embodiment.

FIG. 2 depicts an alternative optical needle probe according to an embodiment.

FIG. 3 depicts a cross-sectional view taken along line III-III of FIG. 1.

FIG. 4 depicts a spectrophotometric system using an optical needle probe according to an embodiment.

FIG. 5 depicts a probe inserted into an excised tumor according to an embodiment.

FIGS. 6A-6C depict display information regarding tissue type as may be provided by an embodiment.

FIG. 7 depicts a tumor ablation procedure according to an embodiment.

FIGS. 8A-8C depict display information regarding tissue type as may be provided by an embodiment.

### DETAILED DESCRIPTION

Cancerous cells have a notably different cellular composition compared to healthy cells since differing concentrations of spectrally active molecules, including metabolites, nucleotides and proteins, lead to characteristic spectral differences. Additionally changes to oxygen content of the tissue are indicative of increased vascularity, which can be determined through the spectral properties of oxyhemoglobin. By illuminating tissue with light across a spectrum which may include near ultra-violet (UV), visible and near infra-red (IR) wavelengths, and measuring diffuse reflected light, essential information about the abundance of certain optically active molecules may be ascertained for the tissue.

By configuring a probe for in vivo analysis, spectrophotometric analysis may be used for monitoring vital signs or diagnosing various conditions within a patient at a crucial time, such as during a surgical procedure. For example, spectrophotometry may be useful for determining oxyhemoglobin, deoxyhemoglobin, cytochrome oxidase, myoglobin, NAD, NADH, NADP, and/or NADPH. Spectrophotometric differences between cancerous cells and healthy cells enable the cells to be distinguished from one another and may be usable therefore during the resection of tumors for on-site verification that proper margins have been used.

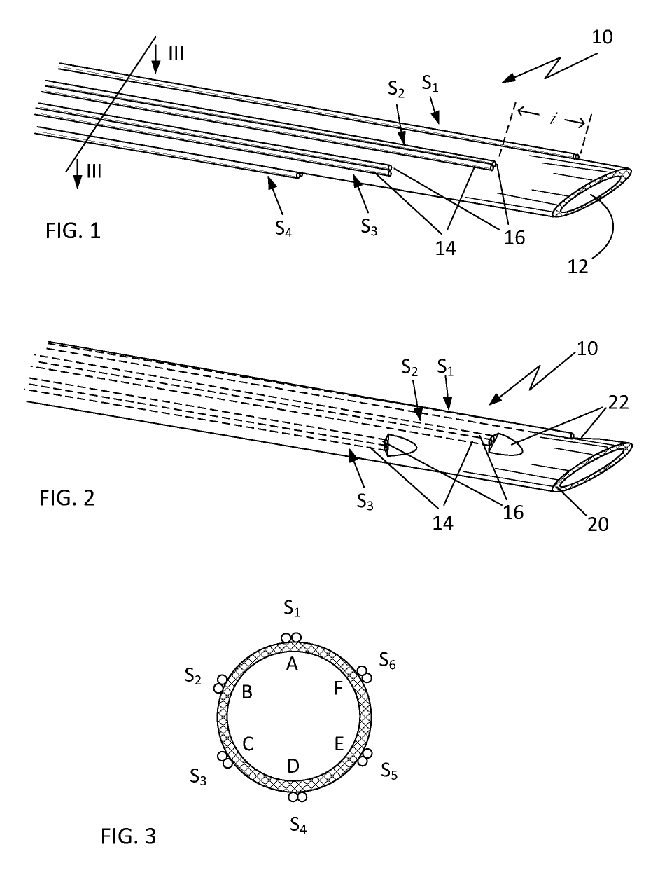

As shown in FIG. 1, a fiber optic probe for in vivo spectrophotometric analysis may be configured as a needle 10 having an internal longitudinal bore 12 which includes multiple sets of optical fibers S₁-Sₙ. The needle may have a diameter from about 0.5 mm (25 gauge) for a general probing needle, to about 3 mm (11 gauge) for a cryoablation probe, or about 0.5 mm to about 2.75 mm (12-24 gauge) for RF ablation probes. These values provide a few examples of sizes for two possible ablation devices. While other devices may be used (for example. thermoablation) the size of such a device may generally fall in the range of 11-24 gauge. The needles may also have diameters greater than, or less than the diameters as stated above. As examples, the needle may have a diameter of about 0.46 mm (26 gauge), about 0.51 mm (25 gauge), about 0.56 mm (24 gauge), about 0.64 mm (23 gauge), about 0.72 mm (22 gauge), about 0.82 mm (21 gauge), about 0.91 mm (20 gauge), about 1.07 mm (19 gauge), about 1.27 mm (18 gauge), about 1.47 mm (17 gauge), about 1.65 mm (16 gauge), about 1.83 mm (15 gauge), about 2.11 mm (14 gauge), about 2.41 mm (13 gauge), about 2.77 mm (12 gauge), about 3.05 mm (11 gauge), about 3.40 mm (10 gauge), about 3.76 mm (9 gauge), about 4.19 mm (8 gauge), about 4.57 mm (7 gauge), about 5.19 mm (4 gauge), about 6.54 mm (2 gauge), or about 8.25 mm (0 gauge) or any diameter between any of the listed values.

Alternatively, in an additional embodiment, the optics may be integrated into an access sheath, which may be a rigid or flexible tube. An access sheath may be inserted into a tissue for the purpose of providing an open access path through which a probe, or other instrument may be inserted. The sheath may be formed of a polymer, and may have any of the above-mentioned sizes, or any size as may be appropriate for the intended purpose of the sheath.

Each set of optic fibers S₁-Sₙ may include at least one light emitting fiber 14, and at least one light collecting fiber 16. Each of the fibers 14, 16 may have a diameter of between about 50 μm to about 200 μm. As examples, the optic fibers may have a diameter of about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, or about 200 μm, or any diameter between any of the listed values, or greater than or less than the listed values. In an embodiment as shown in FIG. 1, the fibers 14, 16 may be placed on the exterior circumferential surface of the needle 10. In an alternative embodiment as represented in FIG. 2, the fibers 14, 16 may be disposed within the wall 20 of the needle, and the wall may have cut-out notches 22 for exposing the ends of the fibers. In an additional embodiment (not shown) the fibers 14, 16 may be disposed along the internal wall, and the wall 22 may have openings for passages of the ends of the fibers to terminate at the external surface of the needle 10.

As depicted in FIGS. 1 and 2, each of the fiber sets S₁-Sₙ may terminate at a different position along the length of the needle 10 to provide a diagnostic reading from each of the terminal ends at numerous locations along the needle. The interval spacing i of terminal ends in the longitudinal direction of the needle 10 may be about 1 mm, 1.5 mm, 2.0 mm, 2.5 mm, 3.0 mm, 3.5 mm, 4.0 mm, 4.5 mm or 5.0 mm, or any desired spacing which may be appropriate for a procedure which is being conducted.

As shown in FIGS. 1 and 2, and with further reference to FIG. 3, the terminal ends of the fiber sets S₁-Sₙ are positioned consecutively in a spiral-type configuration around the needle 10, so that the end of set S₁ is closest to the tip of the needle in position A. The terminal end of set S₂ at position B is next closest to the tip, proceeding consecutively around the needle with the end of set S₆ at position F being the farthest from the tip. In an alternative configuration wherein there may be concern for interference by light from adjacent sets, for example, light from set S₂ being picked up by set S₁, the terminal ends in sequence from the tip may be placed in alternate positions around the circumference to minimize interference. As such, the end of set S₁ may be closest to the tip in position A, and the set S₂ ending next closest to the tip may be located at position D. The set S₃ ending next closest to the tip beyond the end of set S₂ may be located at position B, the set S₄ ending next closest to the tip beyond the end of set S₃ may be located at position E, the set S₅ ending next closest to the tip beyond the end of set S₄ may be located at position F, and the set S₆ ending the farthest from the tip may be located at position C.

In alternative embodiments, the number of sets Sₙ may be less than, or greater than six. As examples, there may be three sets of optical fibers, four sets, five sets, six sets, seven sets, eight sets, nine sets, or ten sets, or essentially any number of sets as may be desired and that may be accommodated by the circumference of the needle 10. In addition, some of the sets may be disposed within the wall 20 as in FIG. 2, and some may be on the exterior as in FIG. 1, to accommodate an even greater number of sets of fibers. Likewise, the terminal positions of the ends of the sets may vary from the configurations as shown and discussed above.

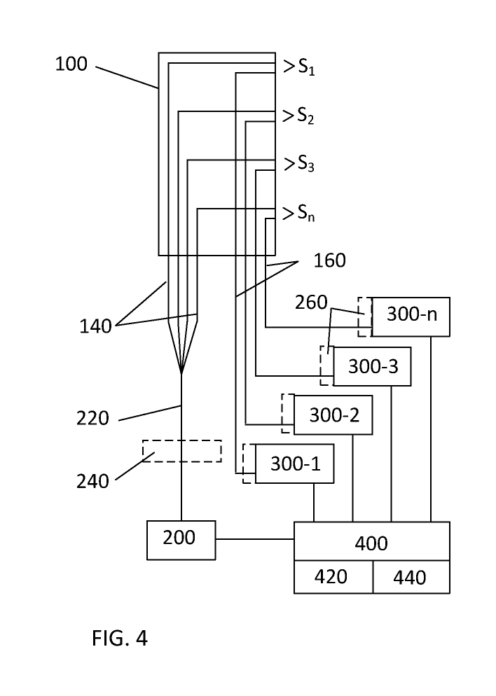

FIG. 4 depicts a schematic illustration of an analysis system which may be used with a needle probe 10. As depicted, the system may include a needle body 100 with emitting fiber optic members 140, and collecting fiber optic members 160 arranged in fiber optic sets S₁-Sₙ. The fiber optic members 140 may be disposed along the needle body 100 in a manner as discussed above to allow radiation of tissue adjacent the needle body. The fiber optic members 140 may be in communication with a light source 200, and the fiber optic members 160 may be in communication with detectors 300-1-300-n.

The light source 200 may be at least one light emitter, a bispectral emitter, a dual spectral emitter, at least one photoemitter, at least one photodiode, at least one light emitting diode, or a semiconductor die. In an embodiment, the light source 200 may be configured as a multiple LED light source, emitting wide band near ultraviolet (UV) through to near infrared (IR) light. The light source 200 may be disposed in any suitable position. For example, the light source 200 may be disposed on the back end of the needle body 100 itself, or, as shown, may be remote of the needle body, and provide illumination via a fiber optic cord 220 extending from the light source to the fiber optic members 140 of the needle body. The emitted light may be passed directly down the cord 220 to the emitting fibers 140, or the light may be passed through an optional monochromator 240 to provide a specific wavelength or a limited range of wavelengths as may be required for a selected function of the system.

Each fiber optic set Sₙ may be connected by means of return fibers 160 to individual light detectors 300-n, to pass light which is either backscattered in reflectance spectrophotometry, or which passes through tissue in transillumination spectrophotometry, back to the detectors. The detectors 300-n may be any type of electromagnetic radiation detecting device, such as, a photoelectric receiver, a photodetector, a photodiode receiver, or a semiconductor die. In an embodiment, the detectors 300-n may be wideband photo detectors or high sensitivity photoresistors. Alternatively, if a multispectral approach may be desired, white light may be emitted from the light source 200, and upon return, the light may be diffracted with optional diffraction devices 260, such as a diffraction grating or prism, before reaching the detectors 300-n, allowing precise analysis across the near UV, visible, and near IR ranges essentially simultaneously.

The light detectors 300-n may be disposed in any suitable position. For example, as shown, the detectors 300-n may be in direct communication with fiber optic members 160, or alternatively, the detectors may be disposed in the needle or adjacent the back end of the needle body 100, or remotely disposed and sense a volume of light through a fiber optic cable or like structure in communication with the needle. The light source 200 and the light detector 300-n communicate with a processing and control unit 400. Unit 400 may comprise any suitable external device, or may be integral with, or immediately adjacent, the needle body 100. Unit 400 may generally be any suitable device for reading, interpolating, evaluating, sensing or using information or phenomena provided to it for calculating, displaying, reading or manipulating the same to allow a user to discern, calculate, interpolate or establish a vita or a condition, or the absence of a condition. In an embodiment, unit 400 may be a spectrophotometer. Unit 400 may include an input device 420, such as a keyboard, and may include an output device 440 such as a monitor for displaying results.

With a system as shown in FIG. 4, changes between emitted and collected light may be determined along the entire length of the needle 100, and the changes may be used to indicate, for example, oxygen content and/or other information regarding tissue condition which may not be as readily attainable with other traditional imaging systems. Additional examples of tissue conditions detectable with such a system are discussed in more detail herebelow.

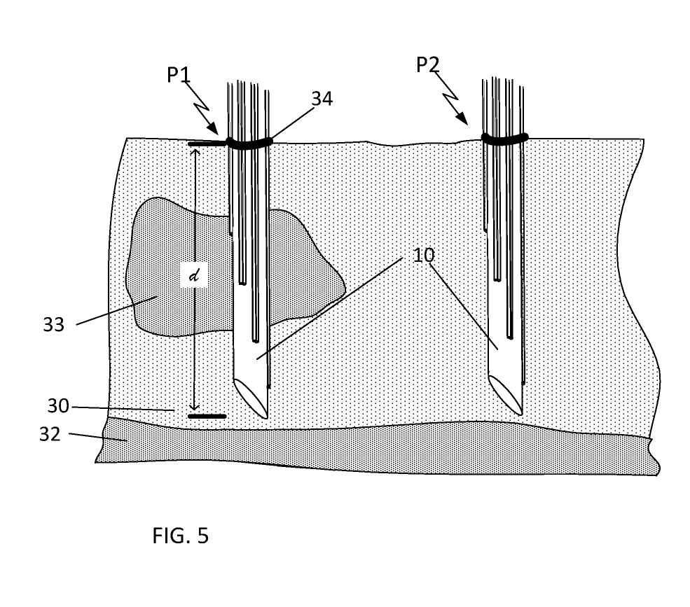

As shown in FIG. 5, a needle 10 of FIG. 1 may be inserted into a tissue specimen which may be an excised cancerous tumor 32 surrounded by a margin of healthy tissue 30 which may contain other cancerous cells 33. The needle 10 may be inserted to a predetermined depth d which may correspond to an acceptable excision margin and which may be indicated by a depth mark 34 on the needle. Monochromatic light, or wide bandwidth light may be supplied and passed into the tissue through the delivery fiber 14, and light reflected by the tissues 30, 32, 33 may be collected by the at least one return fiber 16 and at least the intensity and/or the wavelengths of the return light may be measured. The collected light will provide characteristics of the tissue.

Several different molecular markers may be distinguished using the system, each with a unique signal. Diffuse reflectance of a single wavelength is indicative of tissue absorbance at that wavelength. Upon irradiation, the wavelength may be absorbed directly by a target molecule, or the incident wavelength may generate fluorescent emissions in a target molecule. Measurements may be made either by measuring absorbance or emission, and differing tissue types may be determined based on the differences in the measurements.

One type of measurement which may be conducted is for oxygenation, or oxygen saturation level. In general, methods for non-invasively measuring oxygen saturation utilize the relative difference between the electromagnetic radiation absorption coefficient of deoxyhemoglobin, Hb, and that of oxyhemoglobin, HbO₂. Tissue oxygenation may be measured through absorbance of light of a first wavelength by oxyhemoglobin, in relation to the absorbance of light of a second wavelength by deoxyhemoglobin. In one embodiment, tissue oxygenation may be measured through absorbance of light of approximately 410 nm by oxyhemoglobin, in relation to the absorbance of light of approximately 420 nm by deoxyhemoglobin. In alternate embodiments, additional wavelengths may also be used, as Hb has a second absorption peak at about 580 nm and HbO₂ has additional peaks at about 550 nm and about 600 nm. Additionally, at wavelengths greater than about 600 nm HbO₂ absorption decays much more rapidly than Hb. One or more wavelengths above about 600 nm may be employed either in conjunction with or in place of peak absorption ratios. By using light at these wavelengths, the reflected and returned light is inversely proportional to the quantity of each of the species in the tissue.

Additional absorption wavelengths may also be used, such as the wavelengths used in pulse oximeters. For pulse oximetry one wavelength is about 660 nm and the other is infrared, and may be any one or more of: about 905 nm, about 910 nm, or about 940 nm Absorption at these wavelengths differs significantly between oxyhemoglobin and its deoxygenated form, and may be applied to the optical system as discussed above, and the oxy/deoxyhemoglobin ratio may be calculated from the ratio of the absorption of the red and infrared light.

As mentioned above, fluorescence is also usable for spectral analysis for in vivo tissue diagnosis. By using multiple stimulating wavelengths and measuring emission at a specific wavelength, tissue types can be readily discriminated. As an example, fluorescence ratios may be used to accurately discriminate adipose tissue, healthy tissue and cancerous tissue by measuring emission at 340 nm from excitation at both 289 nm and 271 nm, and measuring emission at 460 nm and also 520 nm with excitation at 340 nm. A large, statistically significant difference was observed between normal, cancerous and adipose tissue. The following table lists representative values of emission ratios for various tissues.

TABLE 1
Summary of the 289/271 excitation ratio from emission
at 340 nm and the 460/520 emission ratio with excitation
at 340 nm for different tissue types.

| | 289/271 | 460/520 |
|---|---|---|
| Normal | 0.509 ± 0.021 | 1.055 ± 0.025 |
| Fat | 0.872 ± 0.097 | 1.314 ± 0.036 |
| Cancerous | 0.445 ± 0.051 | 1.655 ± 0.024 |

Measurements of cancerous tissue oxygen content may provide data on vascularization of the cancerous tissue, while the measured fluorescent ratio may provide data on tissue condition. A combination of these two may therefore allow for an essentially immediate and accurate determination of the state of the tissue.

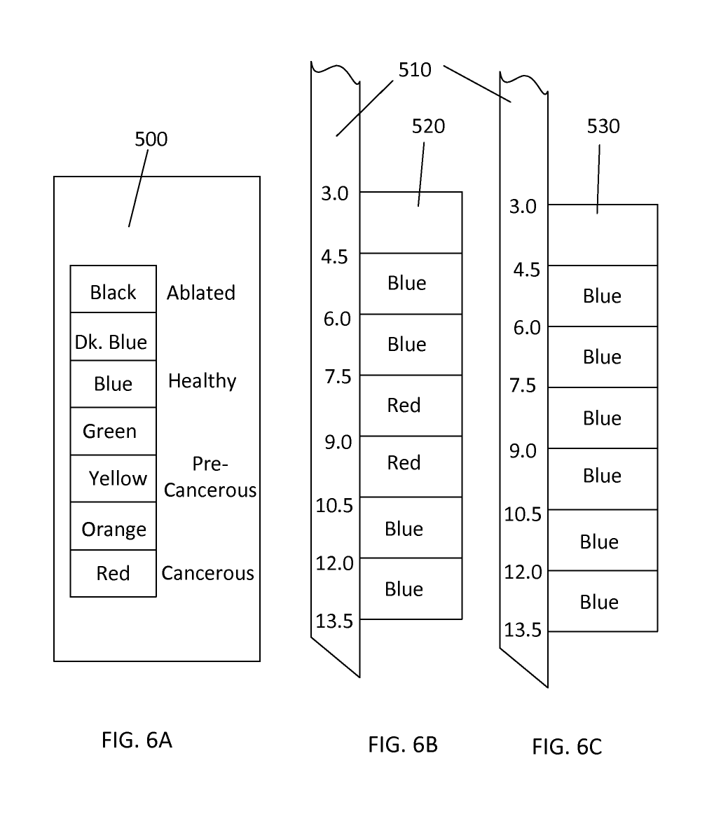

For a rapid display of information on whether or not a certain portion of tissue is cancerous and whether the portion of tissue has been successfully ablated, the processing system 400 of FIG. 4 may be programmed to output a color display representing various tissue types on the monitor 440. One embodiment of a display configuration is illustrated in FIGS. 6A-6C. As depicted in FIG. 6A, a color-key 500 may be used to indicate properties of the tissue, wherein red may indicate cancerous tissue, yellow may indicate precancerous, blue may indicate healthy tissue and black may indicate ablated tissue. As shown in FIGS. 6B and 6C, corresponding to needle positions P1 and P2 of FIG. 5, the monitor screen 440 may show a graphic of a needle 510 with colored shading indicating the state of tissue across the length of the needle. The screen may thereby provide rapidly read information about the state of the tissue the needle is embedded in, and at a glance the position of the needle tip can be verified.

In an alternative embodiment, wherein a surgery team may be using additional monitoring devices such as ultrasound (US) or computed tomography (CT) to display a location of a tumor, for example, a color overlay showing the needle and corresponding tissue readings may be overlaid on the CT or US images so that the surgical team may be able to readily determine the type of tissue which is being displayed on the screen, simultaneously with the additional information being provided by the CT or US.

Following a tumor resection, verification of healthy tissue margin is essential to minimize likelihood of recurrence of the cancer. Pathology verification is the usual standard by which this is done, with the obvious drawbacks of high cost, and long waiting periods. Adding an additional pre-pathology analysis step which verifies that a margin of healthy tissue has been left may allow surgical errors to be immediately rectified, so that a second, corrective procedure may not be needed, reducing total cost of care drastically.

### EXAMPLES

### Example 1

### A Margin Verification Needle Probe

A diagnostic needle for margin verification will be produced. The needle will be an 11 gauge and have a diameter of about 3 mm, corresponding to a circumference of about 9.4 mm Spaced equidistantly and affixed around the circumference will be 10 pairs of fiber optic cables, with each cable having a core diameter of about 100 μm. Each set will have an end terminus on the needle, and the ends will be consecutively spaced from one another, starting at about 1.5 mm from the needle tip, by a distance of 1.5 mm, so that the ends of the pairs will be disposed over a length of about 15 mm from the needle tip. The distal ends of the fiber optics will be provided with fiber optic couplings for connecting one fiber of each pair to a light source, and the other fiber of each pair to a detector.

### Example 2

### Tumor Margin Verification

A needle device 10, such as that of FIG. 1, and as produced by Example 1, with optical fiber sets Sₙ disposed about the needle and terminating at intervals of about 1.5 mm from one another along the length of the needle, will be used to measure margins of healthy tissue around an excised tumor to determine whether additional cancerous tissue may still exist in the required healthy range. The margin for excised tumors may be as little as 1-2 mm and may extend up to about 15 mm depending on the tissue type. By using a spectrophotometric system of FIG. 4, a determination of a safe margin may be rapidly established. With reference to FIG. 5, which may represent an outer edge of an excised tumor 32, a surgeon will wish to determine whether tissue 30, which represents a margin of 15 mm, is clear of cancer cells. In the illustration of FIG. 5, a small tumor mass 33 remains present in the 15 mm margin. The needle 10 will be placed into the excised tissue 30 at a first site P1 up to the required depth d of 15 mm. A depth stop 34 will be mounted on the needle barrel at a distance of 15 mm from the tip, and the needle will be inserted into the excised tissue mass. Measurements of the absorbance ratios A450/A420, and fluorescence ratios at 340 nm, 460 nm and 520 nm will be taken as discussed above and a visual output depicting a representative needle 510 and a color overlay 520, such as that represented in FIG. 6B may be produced on the monitor indicating a cancer presence at a depth of between about 7.5 mm to about 10.5 mm.

The needle 10 will be removed from the excised tissue, placed into another site P2, and further measurements will be taken, and another visual output depicting a representative needle 510 and a color overlay 530, such as that represented in FIG. 6C may be produced on the monitor indicating that the site is clean. This will be repeated at a number of sites along the tissue surface from around the excised tissue. Depending on the tumor size, the number of sites may range from as few as 1 up to 20-30 or more to statistically indicate that the probability of complete cancerous cell removal has been accomplished. Once the needle 10 is inserted, a reading will be displayed instantaneously, providing visual feedback on the condition of the tissue. This procedure may be performed by a nurse, surgeon or pathologist, for example, directly in the operating room and immediately after the excision and prior to closing of the site. As depicted in FIG. 6B, an indication will be provided by the generated red colors to indicate that cancerous cells are still present in the desired safety margin, and the surgeon may perform an additional excision in the appropriate area.

### Example 3

### Surgical Ablation of Tumors

Percutaneous ablation may be used to treat isolated renal tumors or metastasized hepatic tumors in patients where resection is not possible, such as in patients with cirrhosis. Typically, verification of the ablation probe location has been achieved through use of real time computed tomography (CT) imaging. For ablation, extreme accuracy of ablation probe placement is essential to ensure healthy tissue is not unnecessarily damaged. While CT and ultrasound (US) are capable of identifying solid tumor masses, if the tumor has a diffuse edge, positioning the needle so that it removes a clear margin is challenging when viewing via a CT or US image. Detailed and accurate assessment of surrounding tissue is essential to successful treatment without excessively damaging healthy issue. A needle probe 10 as discussed with reference to FIG. 1 may provide a more accurate tissue assessment while performing an ablation.

A needle probe 10 will be used to perform an ablation on a cancerous tumor. The needle probe 10 will be configured to have a bore 12 extending longitudinally therethrough for passage of an ablation implement through the bore. Some examples of ablation implements which may be inserted through the bore 12 include, but are not limited to, a cryoprobe that may be rapidly frozen to freeze surrounding tissue, a radio-frequency antenna to generate RF signals, a fiber optic laser, a heating probe, a rotoablator, a cytotoxic fluid, and/or fluids for enhancing use of any of the above.

In addition, since during performance of an ablation, a single point is essentially targeted, the probe 10 will also be configured so that the terminal end of the fiber optic set S₁ is either co-located with the ablating tip or placed as close as possible to the tip. Additional sets of optic fibers Sₙ will be distributed on the probe 10 so that the ends thereof are placed at intervals up the barrel of the needle (as shown in FIG. 1) to verify correct placement of the needle into unhealthy tissue. Verifying correct needle tip placement in the tumor is essential, and additionally delivering information about surrounding tissue rapidly, both to verify tumor margin and to identify any surrounding precancerous tissue, may provide substantial benefits in attempting to ensure complete ablation.

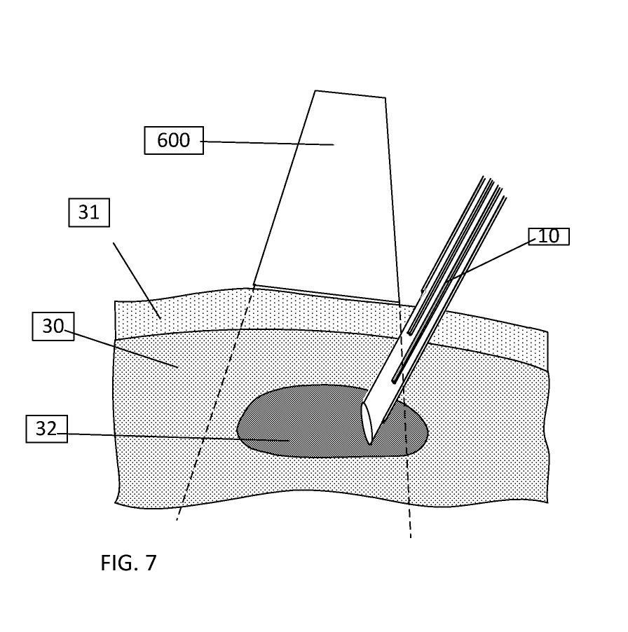

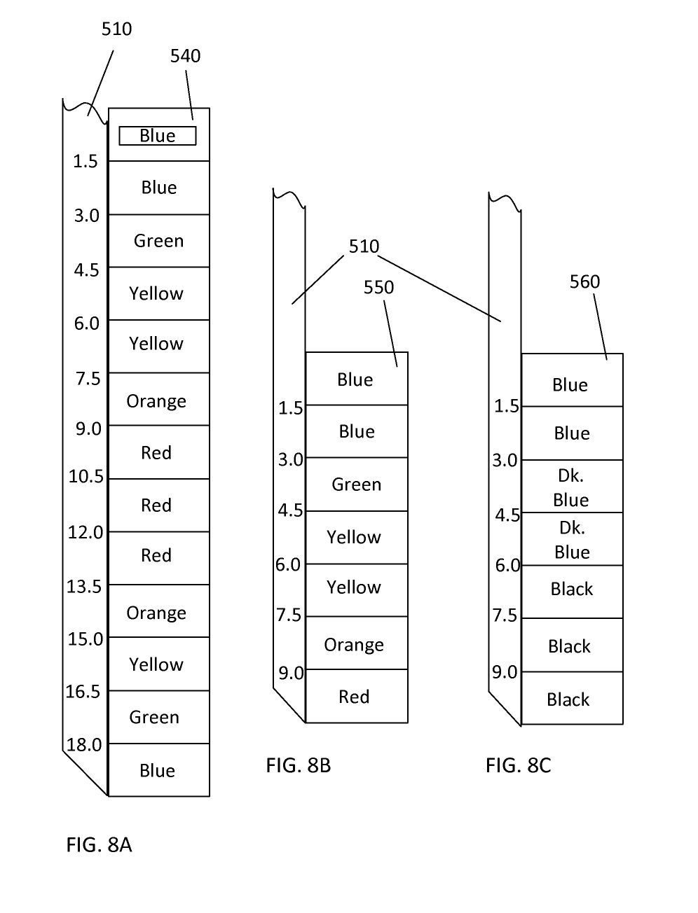

As shown in FIG. 7, an ultrasound transducer placed on the surface of the skin 31 will also be used to guide placement of the needle probe 10. The needle 10 will initially be inserted into tissue 30 and completely through the tumor 32 to determine the extent of the cancer and measure necessary margins. By taking several reading upon insertion or removal, a colored visual display such as depicted by FIG. 8A and showing a depiction of a needle 510 with a color overlay 540 depicting the tissue type may be generated as discussed above. The needle 10 will be withdrawn and reinserted at various locations about the tumor 32 to obtain a three-dimensional representation of the tissue types, borders and required margins. After precisely measuring the margins, both internally and externally of the tumor 32, ablation will be conducted with the additional information provided by the increased data made available to the surgeon by the probe 10.

For the ablation, the needle 10 will be placed so that the tip is in the center of the mass 32 and a display 550 similar to that as represented in FIG. 8B will be displayed on the monitors 440. A radio-frequency ablation will then be conducted by placing an RF-antenna through the bore 12. If needed, the needle tip with RF antenna will be moved through the tumor 32, and withdrawn and reinserted as necessary for ablating the entire tumor. A key aspect to the treatment process will be in monitoring the progress of the tissue ablation by moving and positioning the probe as needed in the tissue, and as the tissue is damaged spectral changes will occur, which can be monitored and reported through the system. A display 560 similar to that as depicted in FIG. 8C will be shown on the monitors indicating that the cancerous cells have been ablated. Regardless of ablation type, measured spectrum will deviate from the initial spectrum in a predictable way, allowing the process of ablation to be monitored throughout the procedure.

This disclosure is not limited to the particular systems, devices and methods described, as these may vary. The terminology used in the description is for the purpose of describing the particular versions or embodiments only, and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1-13. (canceled)

14. A method for intra-operatively determining margins of cancerous tissue during resection of the cancerous tissue, the method comprising:
inserting an optical probe to a predetermined depth into biological tissue to pass through a cancerous tissue therein, the probe comprising:
a shaft having an outer cylindrical surface, a proximal end, and a distal end for being inserted into the biological tissue; and
a plurality of optical fiber sets with each set having a terminal end disposed at a different position relative to the distal end than the terminal end of any other set, and each set comprises at least one light emitting fiber and at least one light collecting fiber;
transmitting light through the light emitting fibers and out the terminal ends thereof into the biological tissue at depths into the biological tissue corresponding to the positions of the ends of the optical fiber sets;
returning light from the biological tissue with the optical fiber sets;
separately detecting light returned through the at least one light collecting fiber of each optical fiber set, wherein the light returned through the at least one light collecting fiber of each optical fiber set comprises at least one property indicative of a type of the biological tissue at the corresponding depths into the biological tissue, the type of tissue being at least one of cancerous tissue, precancerous tissue, fatty tissue, connective tissue or healthy tissue;
displaying the at least one property of the detected light from each optical fiber set to depict whether the biological tissue adjacent the end of each optical fiber set is cancerous tissue, precancerous tissue, fatty tissue, connective tissue or healthy tissue and provide a display for determining margins for the resection;
resecting the cancerous tissue; and
repeating the steps of transmitting, detecting and displaying to determine if any cancerous tissue remains requiring further resection.

15. The method of claim 14, further comprising prior to resecting:
withdrawing the probe from the biological tissue;
reinserting the probe into the biological tissue at a plurality of different locations; and
repeating the steps of transmitting, detecting and displaying at each of the plurality of locations to provide a 3-dimensional mapping of the biological tissue over an area defined by the plurality of different locations for determining the margins for the resection of the cancerous tissue.

16. The method of claim 15, wherein:
the transmitting comprises separately transmitting light having wavelengths of about 271 nm, about 289 nm, and about 340 nm through each optical fiber set;
the at least one property of the light is intensity and the detecting comprises detecting an emission intensity at about 340 nm for the about 271 nm excitation, about 340 nm for the about 289 nm excitation, about 460 nm for the about 340 nm excitation, and about 520 nm for the about 340 nm excitation; and
the method further comprises calculating a ratio of at least one of:
the emission intensity at about 340 nm for the about 271 nm excitation to the emission intensity at about 340 nm for the about 289 nm excitation; and
the emission intensity at about 460 nm for the about 340 nm excitation to the emission intensity at about 520 nm for the about 340 nm excitation,
to determine whether the biological tissue is cancerous tissue, precancerous tissue, fatty tissue, connective tissue or healthy tissue adjacent the end of each set of optical fibers.

17. The method of claim 15, wherein:
the transmitting comprises transmitting light having a range of wavelengths through each optical fiber set into the biological tissue;
the returning comprises returning light having a range of wavelengths from the biological tissue through each optical fiber set to a detection system configured for receiving light having the range of wavelengths and isolating light of at least one wavelength from the range of wavelengths; and
the at least one property indicative of a characteristic of the biological tissue comprises intensity of the light of the at least one wavelength, and the method further comprises measuring the intensity of the light of the at least one wavelength from each fiber optic set to determine whether the biological tissue is cancerous tissue, precancerous tissue, fatty tissue, connective tissue or healthy tissue adjacent the end of each set of optical fibers.

18. The method of claim 17, wherein:
the detection system comprises:
at least one detector corresponding to each at least one wavelength of light; and
at least one wavelength selector for directing the light of the at least one wavelength to its corresponding detector; and
the method further comprises:
receiving the light having the range of wavelengths at the at least one wavelength selector;
directing the at least one wavelength of light to its corresponding detector; and
measuring the intensity of the light of the at least one wavelength of light from each fiber optic set to determine whether the biological tissue is cancerous tissue, precancerous tissue, fatty tissue, connective tissue or healthy tissue adjacent the end of each set of optical fibers.

19. The method of claim 18, wherein the at least one wavelength selector comprises a diffraction grating configured for dispersing the light having the range of wavelengths into a wavelength spectrum and directing at least two different wavelengths of light to their corresponding detectors; and the method further comprises:
dispersing the light having the range of wavelengths into a wavelength spectrum;
directing each of the at least two different wavelengths of light to their corresponding detectors;
measuring the intensity of each of the at least two different wavelengths of light; and
correlating the measured intensities of each of the at least two different wavelengths to determine the type of tissue present adjacent the end of each set of optical fibers.

20-29. (canceled)

30. An optical system for determining at least one property of a material at a plurality of locations within the material, the system comprising:
a needle probe comprising:
a shaft having an outer cylindrical surface;
a proximal end;
a distal end for being inserted into the biological tissue;
a lumen extending from the proximal end to the distal end and being configured for conduction of at least one surgical procedure through the lumen; and
a plurality of sets of optical fibers disposed about the shaft with each set having a terminal end at the outer cylindrical surface and disposed at a different position relative to the distal end than the terminal end of any other set, and each set comprises at least one optical fiber for emitting electromagnetic radiation into the material and at least one optical fiber for collecting and returning electromagnetic radiation from within the tissue;
at least one light source for providing at least a first bandwidth of light to the light transmitting fibers; and
at least one detector for receiving returning light from the at least one light collecting fibers, and outputting at least one signal corresponding to at least one property of the returning light, wherein the at least one property of the returning light from each fiber optic set correlates with at least one property of the material adjacent the end of each fiber optic set.

31. The system of claim 30, wherein the at least one light source comprises a light source capable of emitting light having wavelengths from about 300 nanometers to about 1400 nanometers.

32. The system of claim 31, further comprising a monochromator for selecting a narrow band of wavelengths of the light for transmission through the light emitting fibers.

33. The system of claim 30, further comprising at least one additional light source for providing an alternate bandwidth of light different from the first bandwidth of light.

34. The system of claim 30, wherein the at least one detector comprises at least one of a wideband photodetector and a high sensitivity photoresistor.

35. The system of claim 30, wherein the at least one detector comprises a multispectral detector and the system further comprises at least one device for dispersing light received from each light collecting fiber into a broad spectral band.

36. The system of claim 30, further comprising:
a processing system for receiving the at least one signal corresponding to the at least one property of the returning light from each fiber optic pair, analyzing each signal, and outputting results correlating the at least one property of the received light with the at least one property of the material for the material adjacent the end of each fiber optic pair; and
a display device for displaying the at least one property of the material adjacent the end of each fiber optic pair.

37. The system of claim 30, wherein:
the at least one property of the light comprises intensity of at least one wavelength of the light;
the transmitted light has a first intensity and the returned light has a second intensity;
a decrease of intensity in the returning light correlates with an absorbance of light of the at least one wavelength by an absorbing component; and
an amount of decrease in intensity for each fiber optic pair correlates to a concentration of the absorbing component adjacent the end of each fiber optic pair.

38. The system of claim 30, wherein the material is biological tissue.

39. The system of claim 38, wherein:
the at least one property of the tissue comprises oxygenation;
the at least one light source is configured to provide light having a first wavelength for absorbance by oxyhemoglobin, and light having a second wavelength for absorbance by deoxyhemoglobin;
the at least one detector is configured to measure intensity of the light having the first wavelength and the light having the second wavelength; and
the system further comprises a processing device to:
determine from the measured intensities, an absorbance of light at each of the first and second wavelengths;
calculate oxygenation from a ratio of the absorbance at each of the first and second wavelengths for each fiber optic pair; and
output an oxygenation level for each fiber optic pair.

40. The method of claim 39, wherein the first wavelength of light and the second wavelength of light are a pairing of at least one of: 410 nm and 420 nm; 660 nm and 905 nm; 660 nm and 910 nm, and 660 nm and 940 nm.

41. The system of claim 39, wherein the display device is configured to display a graphical representation of oxygenation at various depths of the probe into the tissue corresponding to the position of the ends of the fiber optic pairs.

42. The system of claim 30, wherein a wavelength of light emitted from the end of the fiber optic pairs is configured to produce an emission of light from the material at a different wavelength, and
an intensity of emission detected for each fiber optic pair correlates with a concentration of an emitting component in the material adjacent the end of each fiber optic pair.

43. The system of claim 30, wherein:
the material comprises biological tissue;
the at least one property of the tissue comprises tissue conditions of normal, cancerous and fatty;
the system is configured to independently emit light having wavelengths of about 271 nm, about 289 nm and about 340 nm from each fiber optic pair;
the at least one detector is configured to measure emission intensity at about 340 nm for the about 271 nm excitation, about 340 nm for the about 289 nm excitation, about 460 nm for the about 340 nm excitation, and about 520 nm for the about 340 nm excitation; and
the system further comprises a processing device to calculate a ratio of at least one of:
the emission intensity at about 340 nm for the about 271 nm excitation to the emission intensity at about 340 nm for the about 289 nm excitation; and
the emission intensity at about 460 nm for the about 340 nm excitation to the emission intensity at about 520 nm for the about 340 nm excitation,
to determine whether the tissue adjacent the end of each fiber optic pair is one of normal, cancerous or fatty.

44. The system of claim 30, wherein the probe comprises a needle and the fiber optic pairs are spaced apart circumferentially around an exterior surface of the needle.

45. The system of claim 44, wherein the end of a first fiber optic pair is disposed at the distal end of the needle, and the end of other fiber optic pairs are disposed at sequentially spaced apart intervals from the distal end.

46. The system of claim 30, wherein the material comprises biological tissue and the lumen is configured to receive a surgical implement therethrough.

47. The system of claim 46, wherein the surgical implement comprises at least one of a cryoprobe, a radio-frequency antenna, a fiber optic laser, a heating probe, a cytotoxic fluid, and fluids for enhancing use of any of the above.

48. The system of claim 30, wherein the display device is configured for receiving information from at least one additional diagnostic source and overlaying the data from the at least one additional diagnostic source with the at least one property of the material for the material adjacent the end of each of the fiber optic pairs.

49-60. (canceled)
